(19) United States
(12) Patent Application Publication
(10) Pub. No.: US 2007/0275020 A1
(43) Pub. Date: Nov. 29, 2007

(54) Method For Hair Treatment With Shape Memory Polymers

(75) Inventors: Andreas Lendlein, Berlin (DE); Axel Kalbfleisch, Darmstadt (DE); Anja Duchscherer, Hadamar (DE); Angelika Beyer, Waldaschaff (DE); Susanne Graner, Buettelborn (DE); Tareq Ali Abdulla Hasson, Aachen (DE); Thomas Krause, Darmstadt (DE); Karl Kratz, Moenchengladbach (DE); Hong-Yan Jiang, Aachen (DE); Simone Moersheim, Eschweiler (DE); Yasmin Ghazlan, Aachen (DE)

Correspondence Address: SEED INTELLECTUAL PROPERTY LAW GROUP PLLC, 701 FIFTH AVE, SUITE 5400, SEATTLE, WA, 98104, US

(73) Assignee: Mnemoscience GmbH, Uebach-Palenberg (DE)

(21) Appl. No.: 10/566,232

(22) PCT Filed: Jul. 14, 2004
(86) PCT No.: PCT/EP04/07801

§ 371 Date: May 17, 2007

(30) Foreign Application Priority Data
Jul. 30, 2003 (DE) 103 34 823.9

(51) Int. Cl. A61K 8/85

(52) U.S. Cl. 424/401; 424/43; 424/70.11; 424/70.16

(57) ABSTRACT

A method for producing a retrievable hairstyling using shape memory polymers is described. Here a composition is applied to the hair, said composition containing a shape memory polymer P or a macromer M that can be cross-linked to a shape memory polymer, the hair is arranged into a given (permanent) shape and said permanent shape is then fixed by means of chemical cross-linking of the macromer M under the formation of a shape memory polymer and/or under physical cross-linking of the polymer P. The polymer P and the macromer M are formed from block copolymers, each of which comprises at least a first block and at least two additional blocks. The first block is a polyol and is selected from polyethers, dimerdiol and derivatives derived therefrom, as well as from polyesters of dicarboxylic acids with diols. The additional blocks are polyesters of hydroxycarboxylic acids or their lactones. The macromer M possesses cross-linkable regions, as well as thermoplastic regions that are not cross-linkable and, after cross-linking, has a transition temperature of Tₜᵣₐₙₛ. The polymer P has a hard segment with a first transition temperature T′ₜᵣₐₙₛ, which is above room temperature, and a soft segment with a second transition temperature Tₜᵣₐₙₛ which is lower than T′ₜᵣₐₙₛ.

The object of the present invention is a method for producing a retrievable hairstyling using shape memory polymers.

In the shaping of hair, a distinction is generally made between temporary and lasting, permanent hair styling. As a rule, a temporary hair styling takes place with the use of compositions based on solutions or dispersions of hair-fixing polymers. Products of this kind give the hair more or less hold, volume, elasticity, springiness and lustre by means of the addition of polymers. For example, in the form of a gel, these styling products facilitate the shaping and creation of a hairstyle; in the form of a hairspray, they improve the condition of a created hairstyle; and, in the form of fixing foams, they increase the volume of the hair. Detrimental is that the desired effects last for only a relatively short time and are quickly lost again as the result of outside influences, such as combing, wind, high air humidity or contact with water. As a rule, a permanent hair styling is accomplished by means of a permanent wave treatment. In this case, disulfide bonds in the hair are reductively cleaved, the hair is brought into a new shape and this new shape is fixed by forming new disulfide bonds by means of an oxidative process. Detrimental is that, as a result of the necessary chemical treatment of the hair with reductive and oxidation agents, it is not possible to avoid damage to the hair structure. A further disadvantage of the methods for hair restyling known up to now is that it is not possible to make the restyling reversible in a relatively simple manner, i.e., to move from one hairstyle to another without complex re-creation.

From JP 04-41416, hair cosmetics are known which contain specific linear polyurethanes having a glass transition temperature T_{g} of from 40-90° C. The method for hair treatment described therein corresponds to a treatment using typical thermoplastic materials. After the application of the composition, a hairstyle is prepared at a temperature above T_{g} and then fixed in place by cooling to below T_{g}. During a subsequent re-heating to a temperature above T_{g} the polymer softens, and a new hairstyle can be prepared. A method for a recallable, reversible hair restyling is not described. The properties of the linear polyurethanes are inadequate for use in retrievable hair styling.

The object underlying the present invention is to provide a method and the products required therefor for a retrievable hair styling with a high degree of recovery for a programmed hairstyle. A further object is to provide a method enabling a permanent hair restyling without damaging intervention in the hair structure. A further object is the provision of a method which enables the reversion of temporary styling shapes multiple times in a simple manner, and which makes it possible to revert to a previously prepared, programmed permanent hairstyle with a high degree of accuracy. A further object is the provision of a method which facilitates, in a simple manner and with a high degree of accuracy, reverse deformations of a hairstyle that have been introduced by external influences and reversion to a previously prepared, programmed permanent hairstyle.

This object is solved by a method for hair treatment, wherein
a composition that contains at least one shape memory polymer P and/or at least one cross-linkable macromer M that forms a shape memory polymer after cross-linking is applied to the hair,

wherein the polymer P and the macromer M are each formed from block polymers having at least a first block which is a polyol selected from polyethers, oligoethers, hydrocarbons having a molecular weight of at least 400 g/mol and at least two alcoholic hydroxyl groups, oligoester diols and polyesters of dicarboxylic acids with diols; and at least two additional blocks, which are polyesters of hydroxycarboxylic acids or their lactones;

wherein

a) the macromer M contains cross-linkable regions that are cross-linkable through chemical bonds and

b) the macromer M contains thermoplastic regions that are not chemically cross-linkable and

c) the shape memory polymer formed after cross-linking has at least one transition temperature Tₜᵣₐₙₛ;

and wherein the polymer P

a) has at least one hard segment that can be cross-linked by means of physical interaction, said hard segment having a first transition temperature T′ₜᵣₐₙₛ which is above room temperature, and

b) has at least one soft segment with a second transition temperature Tₜᵣₐₙₛ which is lower than T′ₜᵣₐₙₛ;
before, simultaneously or subsequently the hair is arranged into a defined (permanent) shape and
the shape is subsequently fixed by chemically cross-linking the macromer, while forming a shape memory polymer and/or using physical cross-linking of the polymer P.

In the context of the invention, a polyol is a compound having at least two alcoholic hydroxyl groups. The hydrocarbons can be linear, branched, saturated, monounsaturated or polyunsaturated. Polyethers and oligoethers are polymer or oligomer compounds whose organic repeating units are held together by ether functionalities (C—O—C). In the context of the invention, polyethers have at least 4 ether groups and at least 5 repeating units, and oligoethers have one, two or three ether groups and from 2 to 4 repeating units.

A further object of the invention is a method for impressing a second hairstyle on to a programmed, retrievable first hairstyle. In this connection, first a hairstyle (permanent shape), programmed by means of the above-mentioned method, is heated to a temperature above Tₜᵣₐₙₛ or between T′ₜᵣₐₙₛ and Tₜᵣₐₙₛ. The hair is then brought into the desired second (temporary) shape and the second shape is fixed by cooling to a temperature below Tₜᵣₐₙₛ.

A further object of the invention is a method for recovery of a first hairstyle (permanent shape) previously programmed by means of the above-mentioned method. For this purpose, a hairstyle in a temporary shape, or a hairstyle deformed by cold forming, is heated to a temperature above Tₜᵣₐₙₛ. The permanent shape forms again spontaneously and automatically.

The invention furthermore relates to a method for reprogramming a permanent hairstyle, previously programmed according to the above-mentioned method, into a different, new permanent shape. For this purpose, the original hairstyle is heated to a temperature above T′ₜᵣₐₙₛ and the hair is brought into a new shape. This new shape is subsequently fixed by means of cooling to a temperature below T′ₜᵣₐₙₛ.

In the context of the invention, the terms “hairstyle” and “shape of the hair” are to be understood broadly and comprise, for example, also the degree of waviness or the degree of straightness of the hair. In the context of the invention, a programmed hairstyle is a group of hairs showing a specific shape due to shape memory polymers which have been cross-linked and fixed in a permanent shape. In the context of the invention, recovery of a programmed hairstyle means that the programmed hairstyle is recovered after a deformation, preferably to a degree of at least 60%, particularly preferably to a degree of at least 80%, in relation to the shape that arises after a first relaxation cycle. The degree of recovery can, for example, be determined by means of measuring the length of a hair curl or hair strand.

Cold-forming of a hairstyle means a change of the hairstyle at the ambient temperature, without the supply of additional heat by means of a hairdryer or similar devices. The deformation can, for example, be caused mechanically, for example, by means of simply hanging the curls under the influence of gravity, by means of combing or brushing the hair, as a result of the wind or humidity, as the result of mechanical influences during sleep or while lying down, etc.

Shape memory polymers in the context of the invention are polymers from which materials can be produced that have the property that they allow any shape to be impressed upon them, to which shape they recover, spontaneously and without the application of external forces, after a deformation or after the impression of a second shape (temporary shape), simply as a result of reheating or another energetic stimulus. At the same time, deformation and recovery are possible multiple times. The degree to which the original, permanent shape is achieved is, as a rule, somewhat less in a first relaxation cycle, consisting of deformation and recovery, than in subsequent cycles, presumably because of the elimination of fault spots, textures, etc. still existing at the beginning. An especially high degree of recovery is then achieved, however, in the subsequent relaxation cycles. The degree of recovery in the first relaxation cycle is preferably at least 30%, particularly preferably at least 50%, and, in the subsequent relaxation cycles, preferably at least 60%, particularly preferably at least 80%. The degree may, however, also be 90% or more. The degree of recovery can be measured in accordance with usual curl retention measurements by simply determining the length of a treated hair curl or by means of known, suitable stress-strain experiments.

In the context of the invention, physically cross-linkable shape memory polymers are polymers with which the fixation of the impressed permanent shape due to cross-linking occurs by physical interactions. A cross-linking by means of physical interactions can be achieved when specific segments of the polymer chains assemble together into crystalline areas. The physical interactions can be charge transfer complexes, hydrogen bonds, dipolar interactions or hydrophobic interactions, Van der Waals interactions or ionic interactions of polyelectrolyte segments. The interactions can occur between different segments within one polymer strand (intramolecular) and/or between different polymer strands (intermolecular). The formation of the interactions can, for example, be initiated by means of cooling (in particular, in cases of crystallisation) and/or by means of drying, i.e., the removal of solvents.

Suitable physically cross-linkable shape memory polymers according to the invention consist of at least one hard segment and at least one soft segment and have at least two transition temperatures Tₜᵣₐₙₛ and T′ₜᵣₐₙₛ. The polymer segments are preferably oligomers or dihydroxy hydrocarbons, in particular, linear chain molecules having a molecular weight of, for example, 400 to 30,000, preferably 1,000 to 20,000 or 1,500 to 15,000. The molecular weight of the polymers can, for example, be from 30,000 to 1,000,000, preferably from 50,000 to 700,000 or from 70,000 to 400,000. These have a degree of crystallinity of preferably from 3 to 80%, particularly preferably from 3 to 60%. The two transition temperatures can, for example, be melting temperatures Tₘ or glass transition temperatures T_{g}. Above Tₜᵣₐₙₛ, the polymer has a lower modulus of elasticity than below Tₜᵣₐₙₛ. The ratio of the moduli of elasticity below and above Tₜᵣₐₙₛ is preferably at least 10, particularly preferably at least 20. The lower transition temperature Tₜᵣₐₙₛ is preferably higher than room temperature (20° C.), in particular at least 30° C., particularly preferably at least 35° C. or at least 40° C., and is that temperature which, when exceeded, sees the spontaneous recovery of the permanent shape from the deformed or temporary shape. Tₜᵣₐₙₛ is preferably so far above the usually to be expected ambient temperatures that at the ambient temperature, no significant, unintentional, thermally induced shaping of the temporary hairstyle occurs. Suitable ranges for Tₜᵣₐₙₛ are, for example, from 25 to 100° C., from 30 to 75° C., from 35 to 70° C. or from 40 to 60° C. The upper transition temperature T′ₜᵣₐₙₛ is higher than Tₜᵣₐₙₛ and is that temperature above which the impression of the permanent shape or the re-impression of a permanent shape into a new permanent shape occurs, and below which the permanent shape is fixed. T′ₜᵣₐₙₛ is preferably so much higher than Tₜᵣₐₙₛ that during the heating of the hairstyle to a temperature above Tₜᵣₐₙₛ for the recovery of the permanent hairstyle or for the reforming of a temporary hairstyle while maintaining the permanent hairstyle, no significant, unintentional, thermally induced deformation of the permanent shape occurs. Preferably, T′ₜᵣₐₙₛ is at least 10° C., particularly preferably at least 20° C. or at least 30° C. above Tₜᵣₐₙₛ. The difference between T′ₜᵣₐₙₛ and Tₜᵣₐₙₛ can, for example, be from 10 to 80° C., from 20 to 70° C. or from 30 to 60° C. Suitable ranges for T′ₜᵣₐₙₛ are, for example, from 40 to 150° C., from 50 to 100° C. or from 70 to 95° C.

The shaping of the hair suitably occurs under heating to a temperature of at least T′ₜᵣₐₙₛ, and the shape of the hair is fixed by means of cooling to a temperature below T′ₜᵣₐₙₛ. Room temperature usually refers to the ambient temperature, preferably at least 20° C. and, in warmer climates, preferably at least 25° C. The application of the composition onto the hair can be carried out in different ways, for example, directly by spraying or indirectly by applying the composition first to the hand or a suitable device, for example a comb, a brush, etc., followed by a subsequent distribution in or onto the hair. The consistency of the composition can, for example, be that of a solution, dispersion, lotion, thickened lotion, gel, foam or a semi-solid compound, creamy or waxy.

Suitable polymers P are, for example, polymers having the following general formula:
A(B)ₙ (I)
wherein A is derived from an n-valent polyether or oligoether, hydrocarbons having a molecular weight of at least 400 g/mol and n alcoholic hydroxyl groups, oligoester diols or from a polyester of a dicarboxylic acid with one diol, or from a dimerdiol, B represents a poly(hydroxycarboxylic acid) block and n represents a number greater than or equal to two, preferably 2, 3 or 4. Preferred are B-A-B triblock polymers, particularly block polymers with a single polyol block as the middle block, two terminal polyester blocks of hydroxycarboxylic acids or their lactones and terminal alcoholic hydroxyl groups.

A preferably represents polyalkylene glycol ethers from polyvalent alcohols, poly(tetrahydrofurane), dimerdiol, dimerdiol oligoethers and oligoester diols (dihydroxy oligoesters). The polyalkylene glycol ethers preferably have from 2 to 6 C atoms per alkylene group, more preferred are polyethylene glycol (PEG) and poly(propylene glycol) (PPG). Dimerdiol is the designation for alpha, omega C36 diols, which can be produced by the dimerization of oleyl alcohol or by the hydrogenation of dimer fatty acids. Dimer fatty acid is a mixture of aliphatic, branched or cyclic C36 dicarboxylic acids (dimer acid), which can be produced by dimerization of oleic acid or tall oil fatty acid (TOFA). Dimerdiol can be obtained, for example, under the trade name Sovermol® 908. Dimerdiol oligoethers are oligomers of dimerdiol, and can be produced by acid-catalyzed dehydration of dimerdiol. Preferred are dimers, trimers and tetramers of dimerdiol. Suitable commercial products are, e.g., Sovermol® 909 with a molecular weight of approximately 1,000 or Sovermol® 910 with a molecular weight of approximately 2,000.

Oligoester diols (dihydroxy oligoesters) are reaction products of an oligomerization of a dicarboxylic acid with a diol, wherein the reaction product has two hydroxyl groups. Suitable dicarboxylic acids are, for example, C3 to C20 dicarboxylic acids, preferably aliphatic C4 to C10 dicarboxylic acids. Aliphatic dicarboxylic acids are, for example, oxalic acid, malonic acid, diacetic acid, glutaric acid, adipic acid, pimelic acid, azelaic acid, sebacic acid, maleic acid or fumaric acid. Aromatic dicarboxylic acids are, for example, phthalic acid and terephthalic acid. Diol components of the oligoester diols are, for example, C2 to C30 diols, preferably aliphatic C5 to C20 diols. Also suitable are glycerol monoesters, particularly monoesters of glycerol with aliphatic C2 to C30 monocarboxylic acids, preferably C5 to C20 monocarboxylic acids.

The poly(hydroxycarboxylic acid) block B can be formed of hydroxycarboxylic acids, particularly monohydroxymonocarboxylic acids having up to 30 C atoms, as well as of their lactones or lactides. The poly(hydroxycarboxylic acid) block B can also be a copolymer of two or more different hydroxycarboxylic acids. Hydroxycarboxylic acids can be: saturated or unsaturated aliphatic hydroxycarboxylic acids, aromatic hydroxycarboxylic acids, alpha-hydroxycarboxylic acids, beta-hydroxycarboxylic acids, omega-hydroxycarboxylic acids or hydroxy fatty acids. Alpha-hydroxycarboxylic acids are, for example, glycolic acid, lactic acid or mandelic acid. Beta-hydroxycarboxylic acids are, for example, beta hydroxyalkane acids, such as beta hydroxybutyric acid or beta-hydroxyvaleric acid. Hydroxy fatty acids are, for example, 12-hydroxystearic acid or ricinoleic acid. Aromatic hydroxy acids are, for example, hydroxybenzoic acids, e.g., salicylic acid.

B preferably stands for poly(ε-caprolactone), polylactides, polyglycolides, poly(lactide-co-glycolide), poly(pentadecalactone), poly(caprolactone-co-lactide), poly(pentadecalactone-co-lactide), poly(pentadecalactone-co-caprolactone). A polyester that is particularly preferred for use according to the invention is a polyester on the basis of lactide units or pentadecalactone units.

Preferred block polymers are those with the general formula:
HO—[B1-C(═O)O—]ₙ₁[Y—O—]ₙ₂[C(═O)—B2-O—]ₙ₃H (Ia)
B1 and B2 are the same or different and stand for branched, cyclic or linear alkylene groups with 1 to 40, preferably 2 to 20 C atoms. Y stands for a branched, cyclic or linear alkylene group with 2 to 30 C atoms, preferably for ethylene groups and/or propylene groups, or for a block comprising dimerdiol, dimerdiol oligoether or oligoester diol, wherein n1, n2 and n3 are the same or different numbers greater than or equal to zero, wherein both n2 and the sum n1+n3 are greater than zero.

Particularly preferred are polymers of the formula (Ia), wherein B1 and B2 stand for branched or linear alkylene groups with 2 to 20 C atoms, Y stands for an ethylene group and where n1, n2 and n3 are greater than zero and are selected in such a way that the molecular weight of the polymer is greater than or equal to 2,000. Some examples of suitable polymers are block copolymers with a polyethylene glycol middle block and terminal blocks of polylactic acid or poly-ε-caprolactone, wherein the terminal blocks are end-substituted with hydroxyl groups, the middle block has a molecular weight in the range from 500 to 20,000, preferably 2,000 to 10,000, and the average molecular weight of the block copolymer is from 2,000 to 50,000, preferably from 3,000 to 25,000.

The advantage of the copolymers with a block structure is that the various properties and functions of the blocks can be combined in one polymer. The properties of the blocks, such as the hydrophilicity, the affinity to hair or the transition temperature, can be selected independently of one another.

For example, to produce the block copolymers, an oligomeric or polymeric diol can be used as difunctional initiator for a ring-opening polymerization (ROP). In this case, the initiator represents the A block. Preferably, polyether diols, which are commercially available in various molecular weights, are used as initiators. Preferred for use are PEO or PEG with a molecular weight of from 4,000 to 8,000 g/mol, particularly preferably with a molecular weight of 6,000 and 8,000 g/mol, which corresponds to the A block length.

Additional preferred diols are:
Dimerdiol (dimer fatty diol), commercially available under the trade name Sovermol® 908, with a molecular weight of 550 g/mol. The production of dimerdiol is known. For example, dimerdiol can be produced by the hydrogenation of dimeric oleic acid and/or its esters according to the German printed patent specification DE-B 17 68 313. The general formula is HO—CH₂—C₃₄H₆₆—CH₂—OH.
Dimerdiol oligoether, commercially available under the trade name Sovermol® 909, with a molecular weight of approximately 1,000 g/mol, and Sovermol® 910, with a molecular weight of approximately 2,000 g/mol. The general formula is HO—CH₂—C₃₄H₆₆—CH₂—O—[CH₂—C₃₄H₆₆—CH₂—O—]ₙH wherein n is a number from 1 to 5, preferably 1, 2 or 3.
Oligoester diols with molecular weights between 1,000 and 6,000 g/mol, which were synthesized from dicarboxylic acids and diols on a fatty basis. The diols on a fatty basis are, in particular, linear or branched aliphatic C2 to C14 diols, 12-hydroxystearyl alcohol, dimer fatty diol, dimer fatty diol oligoethers with a molecular weight of from 1,000 to 2,000 and glycerol monoesters with oleic acid, stearic acid or lauric acid. The dicarboxylic acids are preferably dimer fatty acid, adipic acid and azelaic acid.

The oligoester diols having the following general formulas are suitable:

Oligoester diols of dimerdiol:
HO—CH₂—C₃₄H₆₆—CH₂—O—[(O═)C—R—C(═O)—O—CH₂—C₃₄H₆₆—CH₂—O—]ₙH
Oligoester diols of 12-hydroxystearyl alcohol:
HO—(CH₂)₁₁—CH(C₆H₁₃)—O—[(O═)C—R—C(═O)—O—(CH₂)₁₁—CH(C₆H₁₃)—O—]ₙH
Particularly preferred are hydroxystearyl alcohol-adipate-diol and hydroxystearyl alcohol-azelate-diol.
   Oligoester diols of glycerol monostearate:
   HO—CH(CH₂—O—C(═O)(C₁₇H₃₅))—CH₂O—[(O═)C—R—C—(═O)—O—CH₂—CH—(CH₂O—C(═O)—(C₁₇H₃₅))—CH₂O—]ₙH
   In all of these formulas, R stands for linear, aliphatic hydrocarbons with from 4 to 36 C atoms and n stands for numbers greater than or equal to 1, preferably greater than or equal to 2.

The production of oligoester diols from diols and dicarboxylic acids is known in the state of the art. The production can take place with or without the use of catalysts. All esterification catalysts, such as sulphuric acid, phosphorous acid or p-toluenesulphonic acid can be used as catalysts. Tin compounds, such as tin dioctoate, tin oxide and tin oxalate, and titanium compounds, such as titanium(IV)isopropoxide, can also be considered.

Preferred are p-toluenesulphonic acid and titanium(IV)isopropoxide.

To introduce the B blocks, cyclic esters or diesters can be used, for example, dilactide, diglycolide, p-dioxanone, ε-caprolactone, ω-pentadecalactone or their mixtures. Preferred for use are dilactide, L,L-dilactide or ω-pentadecalactone. Preferably, the reaction takes place in the mass, optionally with the addition of a catalyst, such as dibutyltin(IV)oxide, dibutyltin(IV)dilaurate, tin(II)dilaurate, tin(II)octanoate, titanium(IV)isopropoxide or lithium chloride. Preferred are dibutyltin(IV)oxide and tin(II)octanoate, particularly dibutyltin(IV)oxide. If a catalyst is used, then it can be in the amount of from 0.1 to 0.3 percent by mol. Because many of the possible catalysts, particularly the tin compounds, are toxic, if the triblock copolymers are used in materials for the cosmetic or medical areas, the catalyst residues that remain in the copolymer must be removed. The appropriate operational conditions are known to the expert and illustrated by the following examples.

The B block lengths can be variably adjusted via the molar relationship of the monomer to the initiator. The percent of the weight in A blocks preferably amounts to 40 to 90% in the block copolymers.

Additional suitable block copolymers are, for example, multi-block copolymers, which can be obtained through the reaction of a diol as initiator (block A), first with a first monomer (block B), then with a second monomer (block C).

Suitable block copolymers are, for example, also star-shaped block copolymers, which can be obtained if a triol or tetrol is used as the initiator, instead of the diol. Preferred for use as tri-functional initiators are: commercially available glycerol ethoxylate or glycerol propoxylate or glycerol-propoxylate-b-ethoxylate with a molecular weight of from 200 to 6,000 g/mol, particularly preferred with a molecular weight of 1,000 and 3,000 g/mol. Preferred for use as tetra-functional initiators are: commercially available pentaerythrite-ethoxylate or pentaerythrite-propoxylate or pentaerythrite-propoxylate-b-ethoxylate with a molecular weight of from 200 to 3,000 g/mol, particularly preferred with a molecular weight of 500 and 2,000 g/mol. In principle, diblock copolymers are also suitable.

The described block copolymers are preferably equipped with hydroxyl groups at the terminals. By means of derivatization with a chemically cross-linkable group, terminal groups X can be achieved so that one obtains cross-linkable macromers M, e.g.:
P--->Derivatization--->M
or
A(B)ₙ--->Derivatization--->A(B—X)ₙ
Preferred chemically cross-linkable groups are ethylenically unsaturated, radically polymerizable groups. Radically polymerizable groups are, for example, vinyl groups, allyl groups, acrylate groups or methacrylate groups. For example, they can be introduced by means of esterification with suitable ethylenically unsaturated carboxylic acids or by means of etherification with suitable unsaturated alcohols. Methacrylate terminal groups can be achieved by means of esterification with methacryloyl chloride or methacrylic acid esters, and acrylate terminal groups can be achieved by means of esterification with acryloyl chloride or acrylic acid esters.

In the context of the invention, macromers or pre-polymers which can be cross-linked into shape memory polymers are polymers or oligomers wherein the fixation of an impressed, permanent shape occurs by means of chemical bonds connecting individual polymer strands or oligomer strands. The cross-linkage by means of chemical bonds can be provided by means of ionic or covalent bonds. The cross-linking reaction can be any suitable chemical reaction, for example, a salt formation reaction, a condensation reaction, an addition reaction, a substitution reaction or a reaction induced photochemically or by means of a radical. The cross-linking reaction can take place by using suitable catalysts or initiators, or it can take place without the use of a catalyst. The cross-linking reaction can be initiated by means of a suitable energy source, for example, electromagnetic radiation, ultrasound, heat or mechanical energy. Where necessary, a combination of two or more methods for initiation can be employed in order to increase the efficiency or the speed of the cross-linking reaction.

Covalently cross-linkable shape-memory polymers (macromers M) which are suitable in accordance with the invention possess at least one transition temperature Tₜᵣₐₙₛ. This transition temperature can be a melting temperature Tₘ or a glass transition temperature T_{g}. Above Tₜᵣₐₙₛ, the polymer has a lower modulus of elasticity than below Tₜᵣₐₙₛ. The ratio of the moduli of elasticity below and above Tₜᵣₐₙₛ is preferably at least 20. The transition temperature Tₜᵣₐₙₛ is preferably above room temperature (20° C.), particularly at least 30° C., particularly preferably at least 40° C., and is the temperature above which the spontaneous recovery of the permanent shape occurs, starting from the deformed shape or from the temporary shape.

Suitable macromers M are, for example, macromers having the following general formula:
A(B—X)ₙ (II)
wherein A is derived from an n-valent polyether or oligoether, hydrocarbons having a molecular weight of at least 400 g/mol and n alcoholic hydroxyl groups or oligoester diols or from a polyester of a dicarboxylic acid with one diol, or from a dimerdiol, B represents a poly(hydroxycarboxylic acid) block, n represents a number greater than or equal to two and X represents a reactive, chemically cross-linkable group. A and B can have the same meaning as in formula (I). X is preferably selected from ethylenically unsaturated, radically polymerizable groups, preferably acrylate groups or methacrylate groups, and n is preferably 2, 3 or 4. Preferred are X—B-A-B—X triblock macromers, particularly block polymers with a single polyol block A as the middle block, two terminal polyester blocks B of hydroxycarboxylic acids or their lactones and terminal acrylate groups or methacrylate groups X.

Preferred block copolymers are those with the general formula (IIa):
X1-[O—B1-C(═O)O—]ₙ₁[Y—O—]ₙ₂[C(═O)—B2-O—]ₙ₃X2 (IIa)
As in formula (II), X1 and X2 are the same or different and represent reactive, chemically cross-linkable groups, preferably ethylenically unsaturated, radically polymerizable groups. B1 and B2 are the same or different and stand for branched, cyclic or linear alkylene groups with 1 to 40, preferably 2 to 20, C atoms. Y stands for a branched, cyclic or linear alkylene group with 2 to 30 C atoms, preferably for ethylene groups and/or propylene groups, or for a block comprising dimerdiol, dimerdiol oligoether or oligoester diol, wherein n1, n2 and n3 are the same or different numbers greater than or equal to zero, wherein both n2 and the sum n1+n3 are greater than zero.

Particularly preferred are macromers of the formula (IIa), wherein X1 and X2 are acrylate or methacrylate, B1 and B2 stand for branched or linear alkylene groups with 2 to 20 C atoms, Y stands for an ethylene group and where n1, n2 and n3 are greater than zero and are selected in such a way that the molecular weight of the macromer is greater than or equal to 2,000. Some examples of suitable macromers are block copolymers with a polyethylene glycol middle block and terminal blocks of polylactic acid or poly-ε-caprolactone, wherein the terminal blocks are end-substituted with methacrylate groups, the middle block has a molecular weight in the range from 1,000 to 20,000, preferably 2,000 to 10,000, and the average molecular weight of the block copolymer is from 2,000 to 50,000, preferably from 3,000 to 25,000.

Compositions for the treatment of hair according to the invention contain at least one of the above-mentioned polymers M or cross-linkable macromers M in an amount of preferably 0.01 to 25 percent by weight, particularly preferred from 0.1 to 15 percent by weight, in a suitable liquid medium. The composition can be present as a solution, dispersion, emulsion, suspension or latex. The liquid medium is preferably cosmetically acceptable and physiologically harmless.

In a particularly preferred embodiment, the composition additionally contains macromers that are substituted with only one reactive group. Suitable additional macromers are, for example, macromers of the following general formula:
R—(B′)ₙ—X3 (III)
wherein R designates a monovalent organic residue, X3 designates a reactive, chemically cross-linkable group and —(B′)ₙ— designates a divalent, thermoplastic polymer segment or oligomer segment. X3 is preferably an acrylate group or a methacrylate group. The segment (B′)ₙ preferably represents polyalkylene glycols, their monoalkyl ethers or their block copolymers, wherein the alkylene groups are preferably ethylene groups or propylene groups and wherein the alkyl groups preferably have from 1 to 30 C atoms.

Particularly preferred are terminal polyalkylene glycol monoalkylethers esterified on one end with acrylic acid or methacrylic acid, wherein the alkylene groups are preferably ethylene groups or propylene groups and the alkyl groups are preferably C1 to C30 alkyl groups, for example, poly(ethylene glycol)monoacrylate, poly(propylene glycol)monoacrylate and their monoalkylethers.

The composition according to the invention is generally present in the form of a solution or dispersion in a suitable solution agent. Particularly preferable are aqueous, alcoholic or aqueous-alcoholic solvents. Suitable solvents are, e.g., aliphatic C1 to C4 alcohols or a mixture of water with one of these alcohols. Other organic solvents can, however, also be employed, wherein, in particular, unbranched or branched hydrocarbons, such as pentane, hexane, isopentane; cyclic hydrocarbons such as cyclopentane and cyclohexane; organic linear or cyclic ethers, such as tetrahydrofurane (THF); or liquid organic esters, such as ethylacetate, can be mentioned. Furthermore, solvents on the basis of silicone are also suitable, in particular silicone oils on the basis of linear or cyclic polydimethylsiloxanes (dimethicones or cyclomethicones). The solvents are preferably present in an amount from 0.5 to 99 percent by weight or particularly preferably in an amount from 40 to 90 percent by weight.

The compositions according to the invention can additionally contain from 0.01 to 25 percent by weight of at least one hair care active ingredient, hair-fixative active ingredient and/or hair-colouring active ingredient.

Hair fixatives are, in particular, the known conventional film-forming hair fixative polymers. The film-forming and hair-fixative polymer can be of synthetic origin or of natural origin, and can be of nonionic, cationic, anionic or amphoteric character. Such a polymer additive, which can be present in an amount of from 0.01 to 25 percent by weight, preferably 0.1 to 20 percent by weight, particularly preferably from 0.5 to 15 percent by weight, can also consist of a mixture of more than one polymer, and this additive can be modified further with respect to the hair-fixative properties by means of the addition of further polymers having a thickening effect. Film-forming, hair-fixative polymers according to the invention are polymers which are able, when employed in aqueous, alcoholic or aqueous-alcoholic solutions at a concentration of from 0.01 to 5%, to provide a polymer film on the hair, thereby fixing the hair in this manner.

Homopolymers of vinyl pyrrolidone, homopolymers of N-vinyl formamide, copolymers of vinyl pyrrolidone and vinyl acetate, terpolymers of vinyl pyrrolidone, vinyl acetate and vinyl propionate, polyacrylamides, polyvinyl alcohols or polyethylene glycols having a molecular weight of from 800 to 20,000 g/mol can be employed as suitable synthetic, nonionic, film-forming, hair-fixative polymers in the hair treatment composition according to the invention. Suitable synthetic, anionic, film-forming polymers are crotonic acid/vinyl acetate copolymers and terpolymers of acrylic acid, ethyl acrylate and N-t-butylacrylamide. Natural film-forming polymers or polymers produced therefrom by means of chemical conversion can also be used in the hair treatment composition according to the invention. Low-molecular chitosan with a molecular weight of from 30,000 to 70,000 g/mol or high-molecular chitosan, organo-soluble derivatives of chitosan, mixtures of oligosaccharides, monosaccharides and disaccharides, Chinese balsam resin, cellulose derivatives such as hydroxypropylcellulose having a molecular weight of from 30,000 to 50,000 g/mol, or shellac in a neutralized or unneutralized form have proven satisfactory. Amphoteric polymers can also be employed in the hair treatment composition according to the invention. Suitable are, for example, copolymers of octylacrylamide, t-butylaminoethylmethacrylate and two or more monomers selected from the group consisting of acrylic acid, methacrylic acid and simple esters thereof. Among the cationic polymers that can be used according to the invention, copolymers of vinylpyrrolidone with quaternated derivatives of dialkyl amino acrylate and dialkyl amino methacrylate, such as vinylpyrrolidone/dimethylamino methacrylate copolymers quaternated with diethyl sulphate, can be mentioned. Additional cationic polymers are, for example, the copolymer of vinylpyrrolidone with vinylimidazolium methochloride, the terpolymer from dimethyl diallyl ammonium chloride, sodium acrylate and acrylamide, the terpolymer from vinylpyrrolidone, dimethylamino ethyl methacrylate and vinyl caprolactam, the quaternated ammonium salt produced from hydroxyethylcellulose and an epoxide substituted with trimethylammonium, the vinylpyrrolidone/methacrylamidopropyltrimethyl ammonium chloride copolymer and diquaternary polydimethylsiloxanes.

The consistency of the hair treatment composition according to the invention can be increased by means of the addition of thickeners. In this respect, homopolymers of acrylic acid having a molecular weight of from 2,000,000 to 6,000,000 g/mol are suitable, for example. Copolymers of acrylic acid and acrylamide (sodium salt) having a molecular weight of from 2,000,000 to 6,000,000 g/mol, sclerotium gum and copolymers of acrylic acid and methacrylic acid are also suitable.

A cosmetic composition according to the invention can be used in various forms of application, for example, as a lotion, spray lotion, cream, gel, foam-gel, aerosol spray, non-aerosol spray, aerosol foam, non-aerosol foam, as an o/w emulsion or w/o-emulsion, as a micro emulsion or as a hair wax.

If the composition according to the invention is present in the form of an aerosol spray, it additionally contains 15 to 85 percent by weight, preferably 25 to 75 percent by weight, of a propellant, and is filled into a pressurised container having a spray head. Suitable as propellants are lower alkanes, such as n-butane, isobutene and propane, as well as mixtures thereof, as well as dimethyl ethers or fluorohydrocarbons, such as F 152a (1,1-difluoroethane) or F 134 (tetrafluoroethane), as well as propellants which are present at the pressures in question in gaseous forms, such as N₂, N₂O and CO₂, as well as mixtures of the propellants mentioned in the preceding.

If the hair treatment composition according to the invention is provided in the form of a sprayable non-aerosol hairspray, the composition is sprayed by means of a suitable, mechanical spraying device. Spray devices of the mechanical type are devices which allow the spraying of a composition without using a propellant. Suitable mechanical spray devices are, e.g., spray pumps or elastic containers provided with a spray valve, into which container the cosmetic composition according to the invention is filled under pressure, wherein the elastic container is expanded and wherein the cosmetic composition is continuously discharged if the valve is open, due to the contraction of the elastic container.

If the hair treatment composition according to the invention is provided in the form of a hair foam (mousse), the composition contains at least one conventional foaming substance known in the art for this purpose. The composition is foamed with or without the aid of propellant gases or chemical propellants and worked into the hair as a foam, and then left in the hair without being rinsed out. A product according to the invention has, as an additional component, a device for foaming the composition. Considered as devices for foaming are devices which allow a liquid to be foamed with or without the use of a propellant. For example, a commercially available foam pump or an aerosol foaming head can be used as a suitable mechanical foaming device.

If the hair treatment composition according to the invention is provided in the form of a hair gel, it additionally contains at least one gel-forming substance in an amount of preferably from 0.05 to 10, more preferably from 0.1 to 2, percent by weight. The viscosity of the gel preferably amounts to from 100 to 50,000 mm²/S, more preferably from 1,000 to 15,000 mm²/S, at 25° C., measured as dynamic viscosity using a Bohlin Rheometer CS, measurement body C25 at a shear velocity of 50 s⁻¹.

If the hair treatment composition according to the invention is provided in the form of a hair wax, it additionally contains water-insoluble fatty substances or waxy substances, or substances that provide the composition with a waxy consistency, in an amount of preferably 0.5 to 30 percent by weight. Suitable water-insoluble substances are, for example, emulsifiers having an HLB value of below 7, silicone oils, silicone waxes, wax materials (e.g., wax alcohols, wax acids, wax esters, as well as, in particular, natural waxes such as beeswax, Carnauba wax, etc.), fatty alcohols, fatty acids, fatty acid esters or high-molecular polyethylene glycols having a molecular weight of from 800 to 20,000, preferably from 2,000 to 10,000, g/mol.

If the hair treatment composition according to the invention is provided in the form of a hair lotion, it is present as a substantially non-viscous or low-viscosity solution, dispersion or emulsion, each capable of flowing, with a content of at least 10 percent by weight, preferably from 20 to 95 percent by weight, of a cosmetically compatible alcohol. As alcohols, particularly the lower C1 to C4 alcohols usually used for cosmetic purposes can be used, such as ethanol and isopropanol, for example.

If the hair treatment composition according to the invention is present in the form of a hair cream, it is preferably provided in the form of an emulsion and either it contains additional viscosity-providing components in an amount of from 0.1 to 10 percent by weight or the required viscosity and creamy consistency are built up in the normal way by means of micelle formation with the help of suitable emulsifiers, fatty acids, fatty alcohols, waxes, etc.

In a preferred embodiment, the composition according to the invention is capable of simultaneously facilitating both the impression of a retrievable hairstyle and a provision of hair coloration. The composition is then formulated as a colouring hair treatment composition, e.g., as a colouring fixative, a colouring cream, a colouring foam, etc. It then contains at least one colouring substance. This colouring substance component can be an organic colorant, in particular a so-called direct dye, or it can also involve inorganic pigments.

The total amount of colouring substance in the composition according to the invention is then roughly 0.01 to 7 percent by weight, preferably roughly 0.2 to 4 percent by weight. Suitable direct dyes to be used in the composition according to the invention are, e.g., triphenylmethane colorants, aromatic nitro colorants, azo colorants, quinone colorants or cationic or anionic colorants. Additional known and common colorants for colouring hair that can be used in the colouring substance according to the invention are described in, among other places, E. Sagarin, “Cosmetics, Science and Technology”, Interscience Publishers Inc., New York (1957), pages 503 ff. and H. Janistyn, “Handbuch der Kosmetika und Riechstoffe”, Volume 3 (1973), pages 388 ff. and K. Schrader “Grundlagen und Rezepturen der Kosmetika”, 2^{nd} edition (1989), pages 782-815.

Suitable hair-colouring pigments are practically insoluble colorants in the medium of the composition, and these pigments may be of organic or inorganic nature. Inorganic-organic mixed phase pigments are also possible. The pigments are preferably not nanopigments. The preferred particle size is from 1 to 200 μm, particularly 3 to 150 μm, particularly preferably 10 to 100 μm. Preferred are inorganic pigments.

The hair treatment composition according to the invention preferably additionally contains at least one hair-care substance in an amount of from 0.01 to 10, particularly preferably of from 0.05 to 5, percent by weight. Preferred hair-care substances are silicone compounds, as well as cationic substances which, because of groups that are cationic or that can be rendered cationic, particularly primary, secondary, tertiary or quaternary amine groups, have substantivity to human hair. Suitable cationic substances are selected from cationic tensides, betainic, amphoteric tensides, cationic polymers, silicone compounds with groups that are cationic or that can be rendered cationic, cationically derivatized proteins or protein hydrolysates and betaine.

Suitable silicone compounds are, for example, polydimethylsiloxane (INCI: dimethicone), α-hydro-ω-hydroxypolyoxydimethylsilylene (INCI: dimethiconol), cyclic dimethylpolysiloxane (INCI: cyclomethicone), trimethyl(octadecyloxy)silane (INCI: stearoxytrimethylsilane), dimethylsiloxane/glycol copolymer (INCI: dimethicone copolyol), dimethylsiloxane/aminoalkyl siloxane copolymer having terminal hydroxy groups (INCI: amodimethicone), monomethylpolysiloxane having lauryl side chains and terminal polyoxyethylene chains and/or terminal polyoxypropylene chains (INCI: lauryl methicone copolyol), dimethylsiloxane/glycol copolymer acetate (INCI: dimethicone copolyol acetate), dimethylsiloxane/aminoalkyl siloxane copolymer having terminal trimethylsilyl groups (INCI: trimethylsilylamodimethicone). Preferred silicone polymers are dimethicones, cyclomethicones and dimethiconols. Mixtures of silicone polymers are also suitable, for example a mixture of dimethicone and dimethiconol. The above designations provided in brackets correspond to INCI (International Cosmetic Ingredients) nomenclature employed for the designation of cosmetic active ingredients and additives.

As a rule, additional known cosmetic additives can be added to the hair treatment composition according to the invention, for example non-fixative non-ionic polymers such as polyethylene glycols, non-fixative, anionic and natural polymers, as well as mixtures thereof, in amounts of preferably from 0.01 to 50 percent by weight. It is also possible to add perfume oils in an amount of from 0.01 to 5 percent by weight, opacifiers such as ethylene glycol distearate in an amount of from 0.01 to 5 percent by weight, wetting agents or emulsifiers from the classes of anionic, cationic, amphoteric or non-ionic tensides, such as fatty alcohol sulphates, ethoxylated fatty alcohols, fatty acid alcanolamides, such as esters of hydrated fatty acids from castor oil, in an amount of from 0.1 to 30 percent by weight, as well as moisturizers, dye-receptivity improving agents, light protection agents, anti-oxidative agents and preservatives in amounts of from 0.01 to 10 percent by weight.

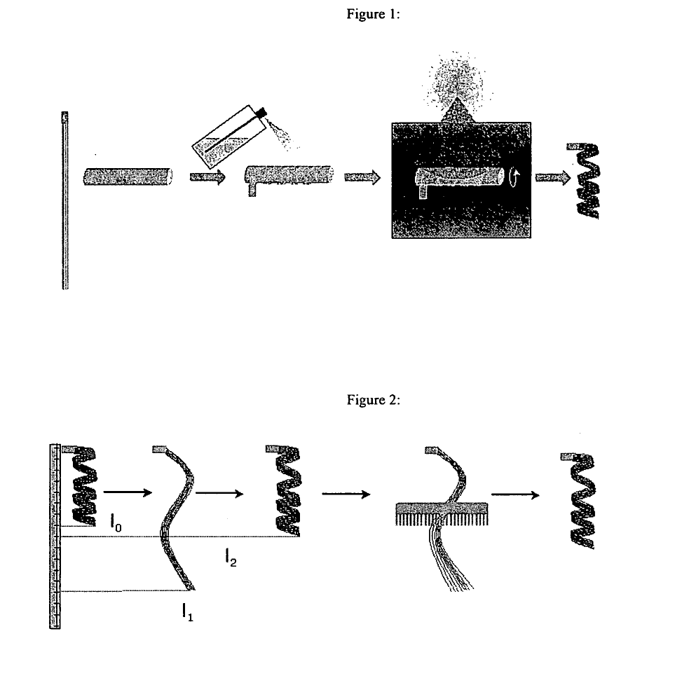

FIG. 1 schematically shows the method for producing a retrievable, permanent hairstyle. A strand of hair is wound onto a hair curler and sprayed with a solution according to the invention that contains a cross-linkable macromer. By irradiation with a suitable source of energy, for example, a UV lamp, the desired permanent shape is fixed. Subsequently the curler is removed.

FIG. 2 shows the deformation of a permanent hairstyle and the recovery of the permanent shape starting from the temporary shape. The hair curl in the permanent shape possesses a length l₀. The curl in the deformed shape has the length l₁. The curl in the recovered shape has the length l₂. The degree of recovery can be calculated as follows: Recovery (l₁−l₂)/(l₁−l₀)

As a measure for the evaluation of the shape memory properties of a composition, it is possible to employ the memory factor, which considers the formability of a permanent hairstyle into a temporary shape (shaping factor), as well as the restoration of the permanent shape, starting from the temporary shape (restoration factor, degree of recovery). If one starts with a straight strand of hair onto which a curly shape has been impressed as a permanent shape and onto which, subsequently, a second, straight shape has been impressed as a temporary shape, the shaping factor can be determined according to the following criteria:

| Degree of straightness | Shaping factor |
|---|---|
| Strongly wavy along the entire length | 0 |
| Slightly wavy from hairline to tip of hair | 1 |
| Hairline straight, tip with curl | 2 |
| Hairline straight, curvature at the tip | 3 |
| Straight all the way from the hairline to the tip | 4 |

The restoration factor can be determined according to the following criteria:

| Degree of restoration of the permanent shape | Restoration factor |
|---|---|
| 0% | 0 |
| 30% | 1 |
| 40% | 2 |
| 50% | 3 |
| 60% | 4 |
| 75% | 5 |
| 100% | 6 |

The memory factor M can be calculated with the following equation using the shaping factor f, the maximum shaping factor F=4, the respective restoration factor r and the maximum restoration factor R=6:
M=(f/F)*(r/R)*100
The memory factor should ideally be not below 20, preferably at least 25 or at least 30, more preferably between 35 and 100.

The following examples are intended to illustrate the subject matter of the invention further.

### EXAMPLES

The production of the triblock copolymers used in the following examples is done by using the diols that form the middle block as initiators and esterification with the respective polyesters that form the terminal blocks using customary esterification reactions. The triblock copolymers substituted with two terminal hydroxyl groups are converted to the macromers substituted with two methacrylate groups with methacryloyl chloride.

Production of an Oligoester Diol

Dicarboxylic acid (1 mol) and diol (1.2 to 1.5 mol) are mixed at 100° C. by stirring. The esterification reaction is then started by adding p-toluenesulphonic acid (1-5 percent by weight, with relation to the total compound). After the end of the reaction, the temperature is cooled to 40° C. and methylene chloride is added to dissolve the reaction mass. The product is precipitated in methanol, washed with methanol and dried in the vacuum-drying chamber. The average molar masses are determined by using GPC (calibration with polystyrene).

### Examples 1 to 112

Compositions, roughly 2-3 percent, of the following block copolymers are produced in ethanol/water (50:50) or ethanol/water/acetone (45:45:10).

| | | | Example no. | |
|---|---|---|---|---|
| | | | Terminal substituents | |
| | | | | Hydroxyl |
| Middle block | Terminal block | Mn | Methacrylate | groups |
| PEG-4k | PDL | 6,000 | 1 | 57 |
| PEG-6k | PDL | 8,000 | 2 | 58 |
| PEG-8k | PDL | 10,000 | 3 | 59 |
| PEG-8k | PDL | 12,000 | 4 | 60 |
| PEG-8k | P-LL-LA | 10,000 | 5 | 61 |
| PEG-8k | P-LL-LA | 9,000 | 6 | 62 |
| PEG-4k | P-DL-LA | 10,000 | 7 | 63 |
| PEG-6k | P-DL-LA | 10,000 | 8 | 64 |
| PEG-8k | P-DL-LA | 10,000 | 9 | 65 |
| PEG-6k | PCL | 8,000 | 10 | 66 |
| PEG-6k | PCL | 10,000 | 11 | 67 |
| PEG-8k | PCL | 10,000 | 12 | 68 |
| PEG-8k | PCL | 12,000 | 13 | 69 |
| PEG-8k | PCL | 15,000 | 14 | 70 |
| PEG-6k | P (CL-co-DL-LA) | 10,000 | 15 | 71 |
| | 30:10 | | | |
| PEG-6k | P (CL-co-DL-LA) | 10,000 | 16 | 72 |
| | 20:20 | | | |
| PEG-6k | P (CL-co-DL-LA) | 10,000 | 17 | 73 |
| | 10:30 | | | |
| PEG-6k | P (CL-co-LL-LA) | 10,000 | 18 | 74 |
| | 30:10 | | | |
| PEG-6k | P (CL-co-LL-LA) | 10,000 | 19 | 75 |
| | 20:20 | | | |
| PEG-6k | P (CL-co-LL-LA) | 10,000 | 20 | 76 |
| | 10:30 | | | |
| PEG-6k | P(CL-co-PDL) 30:10 | 10,000 | 21 | 77 |
| PEG-6k | P(CL-co-PDL) 20:20 | 10,000 | 22 | 78 |
| PEG-6k | P(CL-co-PDL) 10:30 | 10,000 | 23 | 79 |
| PEG-6k | P (LL-LA-co-PDL) | 10,000 | 24 | 80 |
| | 30:10 | | | |
| PEG-6k | P (LL-LA-co-PDL) | 10,000 | 25 | 81 |
| | 20:20 | | | |
| PEG-6k | P (LL-LA-co-PDL) | 10,000 | 26 | 82 |
| | 10:30 | | | |
| PEG-6k | PCL-b-P-DL-LA | 10,000 | 27 | 83 |
| PEG-6k | PCL-b-P-DL-LA | 12,000 | 28 | 84 |
| PEG-6k | PCL-b-P-DL-LA | 15,000 | 29 | 85 |
| PEG-8k | PCL-b-P-DL-LA | 10,000 | 30 | 86 |
| PEG-8k | PCL-b-P-DL-LA | 12,000 | 31 | 87 |
| PEG-8k | PCL-b-P-DL-LA | 15,000 | 32 | 88 |
| PEG-6k | PCL-b-P-LL-LA | 10,000 | 33 | 89 |
| PEG-6k | PCL-b-P-LL-LA | 12,000 | 34 | 90 |
| PEG-6k | PCL-b-P-LL-LA | 15,000 | 35 | 91 |
| PEG-8k | PCL-b-P-LL-LA | 10,000 | 36 | 92 |
| PEG-8k | PCL-b-P-LL-LA | 12,000 | 37 | 93 |
| PEG-8k | PCL-b-P-LL-LA | 15,000 | 38 | 94 |
| PEG-6k | PCL-b-PPDL | 10,000 | 39 | 95 |
| PEG-6k | PCL-b-PPDL | 12,000 | 40 | 96 |
| PEG-6k | PCL-b-PDL | 15,000 | 41 | 97 |
| PEG-8k | PCL-b-PPDL | 10,000 | 42 | 98 |
| PEG-8k | PCL-b-PPDL | 12,000 | 43 | 99 |
| PEG-8k | PCL-b-PDL | 15,000 | 44 | 100 |
| PEG-6k | P-LL-LA-b-PPDL | 10,000 | 45 | 101 |
| PEG-6k | P-LL-LA-b-PPDL | 12,000 | 46 | 102 |
| PEG-6k | P-LL-LA-b-PPDL | 15,000 | 47 | 103 |
| PEG-8k | P-LL-LA-b-PPDL | 10,000 | 48 | 104 |
| PEG-8k | P-LL-LA-b-PPDL | 12,000 | 49 | 105 |
| PEG-8k | P-LL-LA-b-PPDL | 15,000 | 50 | 106 |
| PEG-6k | P-DL-LA-b-PPDL | 10,000 | 51 | 107 |
| PEG-6k | P-DL-LA-b-PPDL | 12,000 | 52 | 108 |
| PEG-6k | P-DL-LA-b-PPDL | 15,000 | 53 | 109 |
| PEG-8k | P-DL-LA-b-PPDL | 10,000 | 54 | 110 |
| PEG-8k | P-DL-LA-b-PPDL | 12,000 | 55 | 111 |
| PEG-8k | P-DL-LA-b-PPDL | 15,000 | 56 | 112 |

### Examples 113 to 200

Compositions, roughly 2-3 percent, of the following block copolymers are produced in ethanol/water/acetone (25:25:50).

| | | | Example no. | |
|---|---|---|---|---|
| Middle | | | Terminal substituents | |
| block | Terminal block | Mn | Methacrylate | Hydroxy |
| Dimerdiol | PPDL | 5,000 | 113 | 157 |
| Dimerdiol | PPDL | 10,000 | 114 | 158 |
| Dimerdiol | P-LL-LA | 5,000 | 115 | 159 |
| Dimerdiol | P-LL-LA | 10,000 | 116 | 160 |
| Dimerdiol | P-DL-LA | 5,000 | 117 | 161 |
| Dimerdiol | P-DL-LA | 10,000 | 118 | 162 |
| Dimerdiol | PCL | 5,000 | 119 | 163 |
| Dimerdiol | PCL | 10,000 | 120 | 164 |
| D-OEt 1000 | PPDL | 5,000 | 121 | 165 |
| D-OEt 1000 | PPDL | 10,000 | 122 | 166 |
| D-OEt 1000 | P-LL-LA | 5,000 | 123 | 167 |
| D-OEt 1000 | P-LL-LA | 10,000 | 124 | 168 |
| D-OEt 1000 | P-DL-LA | 5,000 | 125 | 169 |
| D-OEt 1000 | P-DL-LA | 10,000 | 126 | 170 |
| D-OEt 1000 | PCL | 5,000 | 127 | 171 |
| D-OEt 1000 | PCL | 10,000 | 128 | 172 |
| D-OEt 2000 | PPDL | 5,000 | 129 | 173 |
| D-OEt 2000 | PPDL | 10,000 | 130 | 174 |
| D-OEt 2000 | P-LL-LA | 5,000 | 131 | 175 |
| D-OEt 2000 | P-LL-LA | 10,000 | 132 | 176 |
| D-OEt 2000 | P-DL-LA | 5,000 | 133 | 177 |
| D-OEt 2000 | P-DL-LA | 10,000 | 134 | 178 |
| D-OEt 2000 | PCL | 5,000 | 135 | 179 |
| D-OEt 2000 | PCL | 10,000 | 136 | 180 |
| D-OEs 1000 | PPDL | 10,000 | 137 | 181 |
| D-OEs 1000 | PCL | 10,000 | 138 | 182 |
| D-OEs 1000 | P-LL-LA | 10,000 | 139 | 183 |
| D-OEs 1000 | P-DL-LA | 10,000 | 140 | 184 |
| D-OEs 2000 | PPDL | 10,000 | 141 | 185 |
| D-OEs 2000 | PCL | 10,000 | 142 | 186 |
| D-OEs 2000 | P-LL-LA | 10,000 | 143 | 187 |
| D-OEs 2000 | P-DL-LA | 10,000 | 144 | 188 |
| D-OEs 3000 | PPDL | 10,000 | 145 | 189 |
| D-OEs 3000 | PCL | 10,000 | 146 | 190 |
| D-OEs 3000 | P-LL-LA | 10,000 | 147 | 191 |
| D-OEs 3000 | P-DL-LA | 10,000 | 148 | 192 |
| D-OEs 4000 | PPDL | 10,000 | 149 | 193 |
| D-OEs 4000 | PCL | 10,000 | 150 | 194 |
| D-OEs 4000 | P-LL-LA | 10,000 | 151 | 195 |
| D-OEs 4000 | P-DL-LA | 10,000 | 152 | 196 |
| D-OEs 5000 | PPDL | 10,000 | 153 | 197 |
| D-OEs 5000 | PCL | 10,000 | 154 | 198 |
| D-OEs 5000 | P-LL-LA | 10,000 | 155 | 199 |
| D-OEs 5000 | P-DL-LA | 10,000 | 156 | 200 |

### EXAMPLES

### Hair Treatment with Triblock Copolymers

| | 1 | 2 | 3 | 4 | 5 |
|---|---|---|---|---|---|
| Block copolymer 1 | 3 percent by | | | | |
| | weight | | | | |
| Block copolymer 2 | | 3 percent by | | | |
| | | weight | | | |
| Block copolymer 3 | | | 3 percent by | | |
| | | | weight | | |
| Block copolymer 5 | | | | 3 percent | |
| | | | | by weight | |
| Block copolymer 6 | | | | | 3 percent |
| | | | | | by weight |
| Treatment | UV | UV | UV | UV | UV |
| Solvent | Et/W | Et/W | Et/W | Et/W | Et/W |
| | 50/50 | 50/50 | 50/50 | 50/50 | 50/50 |
| Tₜᵣₐₙₛ [° C.] | 55, 83 | 55-60 | 55-60 | 40, 56 | 40, 56 |
| Memory factor M | 28 | 35 | 33 | 38 | 40 |
| [%] | | | | | |
| | 6 | 7 | 8 | 9 | 10 |
| Block polymer 9 | 3 percent by | | | | |
| | weight | | | | |
| Block polymer 11 | | 3 percent by | | | |
| | | weight | | | |
| Block polymer 15 | | | 3 percent by | | |
| | | | weight | | |
| Block polymer 16 | | | | 3 percent | |
| | | | | by weight | |
| Block polymer 17 | | | | | 3 percent |
| | | | | | by weight |
| Treatment | UV | UV | UV | UV | UV |
| Solvent | Et/W/Ac | Et/W/Ac | Et/W/Ac | Et/W/Ac | Et/W/Ac |
| | 45/45/10 | 45/45/10 | 45/45/10 | 45/45/10 | 45/45/10 |
| Tₜᵣₐₙₛ [° C.] | 50-55 | 50-55 | 50-55 | 50-55 | 50-55 |
| Memory factor M | 30 | 40 | 28 | 28 | 25 |

| | 11 | 12 | 13 | 14 |
|---|---|---|---|---|
| Block copolymer 58 | 3 percent | | | |
| | by weight | | | |
| Block copolymer 61 | | 3 percent | | |
| | | by weight | | |
| Block copolymer 62 | | | 3 percent by weight | |
| Block copolymer 66 | | | | 3 percent by weight |
| Treatment | Heat | Heat | Heat | Heat |
| Solvent | Et/W | Et/W | Et/W | Et/W/Ac |
| | 50/50 | 50/50 | 50/50 | 45/45/10 |
| Tₜᵣₐₙₛ, T′ₜᵣₐₙₛ [° C.] | 57, 85 | 40, 57 | 40, 56 | 44, 53 |
| Memory factor M | 25 | 42 | 40 | 30 |

| | 15 | 16 | 17 | 18 |
|---|---|---|---|---|
| Block copolymer 72 | 3 percent by | | | |
| | weight | | | |
| Block copolymer 115 | | 3 percent by | | |
| | | weight | | |
| Block copolymer 128 | | | 3 percent by | |
| | | | weight | |
| Block copolymer 138 | | | | 3 percent by weight |
| Treatment | Heat | UV | UV | UV |
| Solvent | Et/W | Et/W/Ac | Et/W/Ac | Et/W/Ac |
| | 50/50 | 25/25/50 | 25/25/50 | 25/25/50 |
| Tₜᵣₐₙₛ [° C.] | 50-60 | 50-60 | 50-60 | 50-60 |
| Memory factor M | 40 | 20 | 28 | 30 |

Ac = acetone,
W = water,
Et = ethanol

Hair Coating and Production of the Permanent Shape:

A strand of hair having a length of 20 cm, having been moistened with water, is wound onto a curler and the polymer solution is applied thereon (20 to 30 mg polymer/g hair). The treated strand of hair is then fixed at 70° C. for 30 minutes with or without application of UV light. After cooling to room temperature (about 25° C.), the hair curler was removed. The curled strand (impressed permanent shape) had a length of about 4.5 cm.

Production of the Temporary Shape and Recovery of the Permanent Shape:

In order to produce a temporary shape (e.g., straight), the curled strand is heated to about 55° C., extended to the full original length (20 cm) and cooled again to room temperature. The permanent shape is recovered by heating the straight strand to about 55° C. At this temperature, the strand spontaneously retracts to the permanent (curly) shape.

In order to produce a temporary shape (e.g., straight) again, the curled strand is again heated to 55° C., extended to its full length (20 cm) and cooled to room temperature.

### EXAMPLES OF COSMETIC HAIR COMPOSITIONS

### Example 1

### Aerosol Hair Blower Lotion

| A | | B | | C | | |
|---|---|---|---|---|---|---|
| 3.4 | g | — | | 1.7 | g | Block polymer no. 5 |
| | | 3.4 | g | 1.7 | g | Block polymer no. 61 |
| 0.20 | g | 0.20 | g | 0.20 | g | Perfume |
| 0.02 | g | 0.02 | g | 0.02 | g | Baysilon ® oil PD 5 |
| | | | | | | (phenyl trimethicone) |
| 10.00 | g | 10.00 | g | 10.00 | g | Water |
| Ad 100 | g | Ad 100 | g | Ad 100 | g | Ethanol |

The active ingredient solution is filled into an aerosol can with DME as a propellant, in the ratio 45:55.

### Example 2

### Aerosol Hair Blower Lotion

| A | | B | | C | | |
|---|---|---|---|---|---|---|
| 1.6 | g | — | | 3.2 | g | Block polymer no. 1 |
| 1.6 | g | 3.2 | g | — | | Block polymer no. 57 |
| 3.5 | g | 3.5 | g | 3.5 | g | Amphomer ® LV 71¹⁾ |
| 0.57 | g | 0.57 | g | 0.57 | g | Aminomethyl propanol 95% |
| 0.20 | g | 0.20 | g | 0.20 | g | Perfume |
| 0.02 | g | 0.02 | g | 0.02 | g | Baysilon ® oil PD 5 |
| | | | | | | (phenyl trimethicone) |
| 10.00 | g | 10.00 | g | 10.00 | g | Water |
| Ad 100 | g | Ad 100 | g | Ad 100 | g | Ethanol |

¹⁾Octylacrylamide/acrylic acid/butylaminoethylmethacrylate/methylmethacrylate/hydroxypropyl methacrylate copolymer

The active ingredient solution is filled into an aerosol can with DME as a propellant, in the ratio 45:55.

### Example 3

### Aerosol Hair Blower Lotion

| A | | B | | C | | |
|---|---|---|---|---|---|---|
| 3.4 | g | 1.7 | g | — | | Block polymer no. 66 |
| — | | 1.7 | g | 3.4 | g | Block polymer no. 10 |
| 3.335 | g | 3.335 | g | 3.335 | g | Vinyl acetate/crotonic acid |
| | | | | | | copolymer |
| | | | | | | (Luviset ® CA 66) |
| 0.378 | g | 0.378 | g | 0.378 | g | Aminomethyl propanol 95% |
| 0.20 | g | 0.20 | g | 0.20 | g | Perfume |
| 0.02 | g | 0.02 | g | 0.02 | g | Baysilon ® oil PD 5 |
| | | | | | | (phenyl trimethicone) |
| Ad 100 | g | Ad 100 | g | Ad 100 | g | Ethanol |

The active ingredient solution is filled into an aerosol can with propane/butane (2.7 bar) as a propellant, in the ratio 65:35.

### Example 4

### Hairspray

| | | |
|---|---|---|
| A | B | C |

| 1.25 | g | 2.5 | g | — | | Block polymer no. 65 |
|---|---|---|---|---|---|---|
| 1.25 | g | — | | 2.5 | g | Block polymer no. 9 |
| 3.3 | g | 3.3 | g | 3.3 | g | Luvimer ® 100 p¹⁾ |
| 0.844 | g | 0.844 | g | 0.844 | g | Aminomethyl propanol 95% |
| 0.20 | g | 0.20 | g | 0.20 | g | Perfume |
| 0.02 | g | 0.02 | g | 0.02 | g | Baysilon ® oil PD 5 |
| | | | | | | (phenyl trimethicone) |
| 10.00 | g | 10.00 | g | 10.00 | g | Water |
| Ad 100 | g | Ad 100 | g | Ad 100 | g | Ethanol |

¹⁾t-butyl acrylate/ethyl acrylate/methacrylic acid copolymer

The active ingredient solution is filled into an aerosol can with DME as a propellant, in the ratio 45:55.

### Example 5

### Aerosol Fixing Foam

| A | | B | | C | | |
|---|---|---|---|---|---|---|
| 6 | g | 3 | g | — | | Block polymer no. 63 |
| — | | 3 | g | 6 | g | Block polymer no. 7 |
| 11.9 | g | 11.9 | g | 11.9 | g | Ethanol |
| 0.14 | g | 0.14 | g | 0.14 | g | Celquat ® L200¹⁾ |
| 0.2 | g | 0.2 | g | 0.2 | g | Laureth-4 |
| 0.2 | g | 0.2 | g | 0.2 | g | Panthenol |
| 0.2 | g | 0.2 | g | 0.2 | g | Perfume |
| 0.07 | g | 0.07 | g | 0.07 | g | Cetyl trimethyl ammonium |
| | | | | | | bromide |
| 4 | g | 4 | g | 4 | g | Propane |
| 4 | g | 4 | g | 4 | g | Butane |
| Ad 100 | g | Ad 100 | g | Ad 100 | g | Water |

¹⁾Copolymer of hydroxyethylcellulose and diallyl dimethyl ammonium chloride; polyquaternium-4

### Example 6

### Aerosol Fixing Foam

| A | | B | | C | | |
|---|---|---|---|---|---|---|
| 3 | g | 6 | g | — | | Block polymer no. 62 |
| 3 | g | — | | 6 | g | Block polymer no. 6 |
| 11.9 | g | 11.9 | g | 11.9 | g | Ethanol |
| 0.3 | g | 0.3 | g | 0.3 | g | Gafquat ® 755 N¹⁾ |
| 0.2 | g | 0.2 | g | 0.2 | g | Laureth-4 |
| 0.2 | g | 0.2 | g | 0.2 | g | Panthenol |
| 0.2 | g | 0.2 | g | 0.2 | g | Perfume |
| 0.07 | g | 0.07 | g | 0.07 | g | Cetyl trimethyl ammonium |
| | | | | | | bromide |
| 4 | g | 4 | g | 4 | g | Propane |
| 4 | g | 4 | g | 4 | g | Butane |
| Ad 100 | g | Ad 100 | g | Ad 100 | g | Water |

¹⁾Vinylpyrrolidone/dimethylamino ethyl methacrylate copolymer

### Example 7

### Aerosol Fixing Foam

| A | | B | | C | | |
|---|---|---|---|---|---|---|
| 2 | g | 4 | g | — | | Block polymer no. 64 |
| 2 | g | — | | 4.1 | g | Block polymer no. 8 |
| 0.6 | g | 0.6 | g | 0.6 | g | PVP/Vinyl caprolactam/ |
| | | | | | | DMAPA acrylates |
| | | | | | | copolymer |
| | | | | | | (Aquaflex ® SF 40) |
| 0.07 | g | 0.07 | g | 0.07 | g | Aminomethyl propanol 95% |
| 18.9 | g | 18.9 | g | 18.9 | g | Ethanol |
| 0.4 | g | 0.4 | g | 0.4 | g | PEG 25 PABA |
| 0.2 | g | 0.2 | g | 0.2 | g | Laureth-4 |
| 0.2 | g | 0.2 | g | 0.2 | g | Panthenol |
| 0.2 | g | 0.2 | g | 0.2 | g | Perfume |
| 0.07 | g | 0.07 | g | 0.07 | g | Cetyl trimethyl ammonium |
| | | | | | | chloride |
| 4 | g | 4 | g | 4 | g | Propane |
| 4 | g | 4 | g | 4 | g | Butane |
| Ad 100 | g | Ad 100 | g | Ad 100 | g | Water |

### Example 8

### Aerosol Fixing Foam

| A | | B | | C | | |
|---|---|---|---|---|---|---|
| 2.2 | | 5.5 | | — | | Block polymer no. 60 |
| 2.2 | | — | | 5.5 | g | Block polymer no. 4 |
| 8.9 | g | 8.9 | g | 8.9 | g | Ethanol |
| 0.45 | g | 0.45 | g | 0.45 | g | Polyvinylpyrrolidone (PVP |
| | | | | | | K 90) |
| 0.4 | g | 0.4 | g | 0.4 | g | PEG 25 PABA |
| 1.0 | g | 1.0 | g | 1.0 | g | Abilquat ® 3272 |
| | | | | | | (Quaternium-80, 50%, in |
| | | | | | | propylene glycol) |
| 0.15 | g | 0.15 | g | 0.15 | g | Betaine |
| 0.2 | g | 0.2 | g | 0.2 | g | Perfume |
| 0.07 | g | 0.07 | g | 0.07 | g | Cetyl trimethyl ammonium |
| | | | | | | chloride |
| 4 | g | 4 | g | 4 | g | Propane |
| 4 | g | 4 | g | 4 | g | Butane |
| Ad 100 | g | Ad 100 | g | Ad 100 | g | Water |

### Example 9

### Aerosol Fixing Foam

| A | | B | | C | | |
|---|---|---|---|---|---|---|
| 1.8 | | 3.5 | | — | | Block polymer no. 62 |
| 1.8 | | — | | 3.5 | g | Block polymer no. 6 |
| 8.9 | g | 8.9 | g | 8.9 | g | Ethanol |
| 0.45 | g | 0.45 | g | 0.45 | g | Polyvinylpyrrolidone |
| | | | | | | (PVP K 90) |
| 0.4 | g | 0.4 | g | 0.4 | g | PEG 25 PABA |
| 1.0 | g | 1.0 | g | 1.0 | g | Abilquat ® 3272 |
| | | | | | | (Quaternium-80, 50%, in |
| | | | | | | propylene glycol) |
| 0.2 | g | 0.2 | g | 0.2 | g | Perfume |
| 0.07 | g | 0.07 | g | 0.07 | g | Cetyl trimethyl ammonium |
| | | | | | | chloride |
| 4 | g | 4 | g | 4 | g | Propane |
| 4 | g | 4 | g | 4 | g | Butane |
| Ad 100 | g | Ad 100 | g | Ad 100 | g | Water |

### Example 10

### Aerosol Fixing Foam

| A | | B | | C | | |
|---|---|---|---|---|---|---|
| 2.8 | g | 5.5 | g | — | | Block polymer no. 61 |
| 2.8 | g | — | | 5.5 | g | Block polymer no. 5 |
| 8.9 | g | 8.9 | g | 8.9 | g | Ethanol |
| 0.45 | g | 0.45 | g | 0.45 | g | Polyvinylpyrrolidone |
| | | | | | | (PVP K 30) |
| 0.4 | g | 0.4 | g | 0.4 | g | PEG 25 PABA |
| 0.3 | g | 0.3 | g | 0.3 | g | Celquat ® L200¹⁾ |
| 0.2 | g | 0.2 | g | 0.2 | g | Perfume |
| 0.07 | g | 0.07 | g | 0.07 | g | Cetyl trimethyl ammonium |
| | | | | | | bromide |
| 4 | g | 4 | g | 4 | g | Propane |
| 4 | g | 4 | g | 4 | g | Butane |
| Ad 100 | g | Ad 100 | g | Ad 100 | g | Water |

¹⁾Copolymer of hydroxyethylcellulose and diallyl dimethyl ammonium chloride; polyquaternium-4

### Example 11

### Non-Aerosol Hair Blower Lotion

| A | | B | | C | | |
|---|---|---|---|---|---|---|
| 2.3 | g | 4.5 | g | — | | Block polymer no. 65 |
| 2.3 | g | — | | 4.5 | g | Block polymer no. 9 |
| 0.45 | g | 0.45 | g | 0.45 | g | Aristoflex ® A¹⁾ |
| 27 | g | 27 | g | 27 | g | Ethanol |
| 0.7 | g | 0.7 | g | 0.7 | g | PEG 25 PABA |
| 0.35 | g | 0.35 | g | 0.35 | g | Panthenol |
| 0.1 | g | 0.1 | g | 0.1 | g | Betaine |
| 0.25 | g | 0.25 | g | 0.25 | g | Perfume |
| 0.21 | g | 0.21 | g | 0.21 | g | PEG 40 hydrogenated castor |
| | | | | | | oil |
| 0.20 | g | 0.20 | g | 0.20 | g | Cetyl trimethyl ammonium |
| | | | | | | chloride |
| Ad 100 | g | Ad 100 | g | Ad 100 | g | Water |

¹⁾Vinyl acetate/crotonic acid/polyethylene oxide copolymer

The composition can be filled into a package with a pump spray device and used as a non-aerosol spray.

### Example 12

### Non-Aerosol Hair Blower Lotion

| A | | B | | C | | |
|---|---|---|---|---|---|---|
| 2.3 | g | 4.5 | g | — | | Block polymer no. 62 |
| 2.3 | g | — | | 4.5 | g | Block polymer no. 6 |
| 20 | g | 20 | g | 20 | g | Ethanol |
| 0.7 | g | 0.7 | g | 0.7 | g | PEG 25 PABA |
| 0.35 | g | 0.35 | g | 0.35 | g | Panthenol |
| 0.1 | g | 0.1 | g | 0.1 | g | Betaine |
| 0.25 | g | 0.25 | g | 0.25 | g | Perfume |
| 0.21 | g | 0.21 | g | 0.21 | g | PEG 40 hydrogenated castor |
| | | | | | | oil |
| 0.20 | g | 0.20 | g | 0.20 | g | Cetyl trimethyl ammonium |
| | | | | | | bromide |
| Ad 100 | g | Ad 100 | g | Ad 100 | g | Water |

The composition can be filled into a package with a pump spray device and used as a non-aerosol spray.

### Example 13

### Non-Aerosol Hair Blower Lotion

| A | | B | | C | | |
|---|---|---|---|---|---|---|
| 2.3 | g | 4.5 | g | — | | Block polymer no. 65 |
| 2.3 | g | — | | 4.5 | g | Block polymer no. 9 |
| 0.2 | g | 0.2 | g | 0.2 | g | Polyquaternium-46 |
| | | | | | | (Luviquat Hold) |
| 27 | g | 27 | g | 27 | g | Ethanol |
| 0.7 | g | 0.7 | g | 0.7 | g | PEG 25 PABA |
| 0.35 | g | 0.35 | g | 0.35 | g | Panthenol |
| 0.1 | g | 0.1 | g | 0.1 | g | Betaine |
| 0.25 | g | 0.25 | g | 0.25 | g | Perfume |
| 0.21 | g | 0.21 | g | 0.21 | g | PEG 40 hydrogenated castor |
| | | | | | | oil |
| 0.20 | g | 0.20 | g | 0.20 | g | Cetyl trimethyl ammonium |
| | | | | | | chloride |
| Ad 100 | g | Ad 100 | g | Ad 100 | g | Water |

The composition can be filled into a package with a pump spray device and used as a non-aerosol spray.

### Example 14

### Non-Aerosol Hair Blower Lotion

| A | | B | | C | | |
|---|---|---|---|---|---|---|
| 1.6 | g | 3.1 | g | — | | Block polymer no. 69 |
| 1.6 | g | — | | 3.1 | g | Block polymer no. 13 |
| 0.05 | g | 0.05 | g | 0.05 | g | Celquat ® L200 |
| 0.5 | g | 0.5 | g | 0.5 | g | Polyvinylpyrrolidone/vinyl |
| | | | | | | acetate copolymer |
| 27 | g | 27 | g | 27 | g | Ethanol |
| 0.7 | g | 0.7 | g | 0.7 | g | PEG 25 PABA |
| 0.35 | g | 0.35 | g | 0.35 | g | Panthenol |
| 0.1 | g | 0.1 | g | 0.1 | g | Betaine |
| 0.25 | g | 0.25 | g | 0.25 | g | Perfume |
| 0.21 | g | 0.21 | g | 0.21 | g | PEG 40 hydrogenated castor |
| | | | | | | oil |
| 0.20 | g | 0.20 | g | 0.20 | g | Cetyl trimethyl ammonium |
| | | | | | | bromide |
| Ad 100 | g | Ad 100 | g | Ad 100 | g | Water |

The composition can be filled into a package with a pump spray device and used as a non-aerosol spray.

### Example 15

### Non-Aerosol Hair Blower Lotion

| A | | B | | C | | |
|---|---|---|---|---|---|---|
| 2 | g | 4.1 | g | — | | Block polymer no. 62 |
| 2, | g | — | | 4.1 | g | Block polymer no. 6 |
| 1.0 | g | 1.0 | g | 1.0 | g | Block polymer no. 9 |
| 0.15 | g | 0.15 | g | 0.15 | g | Polyquaternium-46 |
| | | | | | | (Luviquat Hold) |
| 27 | g | 27 | g | 27 | g | Ethanol |
| 0.7 | g | 0.7 | g | 0.7 | g | PEG 25 PABA |
| 0.35 | g | 0.35 | g | 0.35 | g | Panthenol |
| 0.25 | g | 0.25 | g | 0.25 | g | Perfume |
| 0.21 | g | 0.21 | g | 0.21 | g | PEG 40 hydrogenated castor |
| | | | | | | oil |
| 0.20 | g | 0.20 | g | 0.20 | g | Cetyl trimethyl ammonium |
| | | | | | | chloride |
| Ad 100 | g | Ad 100 | g | Ad 100 | g | Water |

The composition can be filled into a package with a pump spray device and used as a non-aerosol spray.

### Example 16

### Non-Aerosol Hair Blower Lotion

| A | | B | | C | | |
|---|---|---|---|---|---|---|
| 3 | g | 6 | g | — | | Block polymer no. 62 |
| 3 | g | — | | 6.0 | g | Block polymer no. 6 |
| 2.0 | g | 2.0 | g | 2.0 | g | Vinyl caprolactam/VD/ |
| | | | | | | dimethylamino ethyl |
| | | | | | | methacrylate copolymer |
| | | | | | | (Advantage ® S) |
| 28.5 | g | 28.5 | g | 28.5 | g | Ethanol |
| 0.25 | g | 0.25 | g | 0.25 | g | Perfume |
| 0.20 | g | 0.20 | g | 0.20 | g | Cetyl trimethyl ammonium |
| | | | | | | chloride |
| Ad 60 | g | Ad 60 | g | Ad 60 | g | Water |

The composition can be filled into a package with a pump spray device and used as a non-aerosol spray.

### Example 17

### Spray Fixer

| A | | B | | C | | |
|---|---|---|---|---|---|---|
| 3 | g | 6 | g | — | | Block polymer no. 62 |
| 3 | g | — | | 6.0 | g | Block polymer no. 6 |
| 2.0 | g | 2.0 | g | 2.0 | g | Amphomer ® |
| 28.5 | g | 28.5 | g | 28.5 | g | Ethanol |
| 0.6 | g | 0.6 | g | 0.6 | g | Aminomethyl propanol 95% |
| 0.25 | g | 0.25 | g | 0.25 | g | Perfume |
| 0.20 | g | 0.20 | g | 0.20 | g | Cetyl trimethyl ammonium |
| | | | | | | bromide |
| Ad 60 | g | Ad 60 | g | Ad 60 | g | Water |

The composition can be filled into a package with a pump spray device and used as a non-aerosol spray.

### Example 18

### Spray Fixer

| A | | B | | C | | |
|---|---|---|---|---|---|---|
| 1.5 | g | 3 | g | — | | Block polymer no. 62 |
| 1.5 | g | — | | 3.0 | g | Block polymer no. 6 |
| 0.65 | g | 0.65 | g | 0.65 | g | Amphomer ® |
| 0.2 | g | 0.2 | g | 0.2 | g | Celquat ® L200 |
| 28.5 | g | 28.5 | g | 28.5 | g | Ethanol |
| 0.6 | g | 0.6 | g | 0.6 | g | Aminomethyl propanol 95% |
| 0.25 | g | 0.25 | g | 0.25 | g | Perfume |
| 0.20 | g | 0.20 | g | 0.20 | g | Cetyl trimethyl ammonium |
| | | | | | | chloride |
| Ad 60 | g | Ad 60 | g | Ad 60 | g | Water |

The composition can be filled into a package with a pump spray device and used as a non-aerosol spray.

### Example 19

### Spray Gel

| A | | B | | C | | |
|---|---|---|---|---|---|---|
| 2.6 | g | 5.2 | g | — | | Block polymer no. 63 |
| 2.6 | g | — | | 5.2 | g | Block polymer no. 7 |
| 20 | g | 20 | g | 20 | g | Ethanol |
| 0.1 | g | 0.1 | g | 0.1 | g | Aminomethyl propanol 95% |
| 0.2 | g | 0.2 | g | 0.2 | g | PEG-40 |
| 0.2 | g | 0.2 | g | 0.2 | g | Perfume |
| 1.5 | g | 1.5 | g | 1.5 | g | Carbomer |
| Ad 70 | g | Ad 70 | g | Ad 70 | g | Water |

The composition can be filled into a package with a pump spray device and used as a non-aerosol spray.

### Example 20

### Spray Gel

| A | | B | | C | | |
|---|---|---|---|---|---|---|
| 2 | g | 4 | g | — | | Block polymer no. 52 |
| 2 | g | — | | 4.0 | g | Block polymer no. 6 |
| 3.0 | g | 3.0 | g | 3.0 | g | VP/VA copolymer (Luviskol |
| | | | | | | VA 64) |
| 18 | g | 18 | g | 18 | g | Ethanol |
| 0.1 | g | 0.1 | g | 0.1 | g | Aminomethyl propanol 95% |
| 0.2 | g | 0.2 | g | 0.2 | g | PEG-40 |
| 0.2 | g | 0.2 | g | 0.2 | g | Perfume |
| 1.5 | g | 1.5 | g | 1.5 | g | Carbomer |
| Ad 70 | g | Ad 70 | g | Ad 70 | g | Water |

The composition can be filled into a package with a pump spray device and used as a non-aerosol spray.

### Example 21

### Pump Fixing Foam

| | | |
|---|---|---|
| A | B | C |

| 2.8 | g | 5.5 | g | — | | Block polymer no. 52 |
|---|---|---|---|---|---|---|
| 2.8 | g | — | | 5.5 | g | Block polymer no. 6 |
| 8.9 | g | 8.9 | g | 8.9 | g | Ethanol |
| 0.2 | g | 0.2 | g | 0.2 | g | Cocamidopropyl |
| | | | | | | hydroxysultaine |
| 0.2 | g | 0.1 | g | 0.2 | g | Cetyl trimethyl ammonium |
| | | | | | | chloride |
| 0.15 | g | 0.15 | g | 0.15 | g | Perfume |
| 0.1 | g | 0.1 | g | 0.1 | g | Citric acid |
| 0.1 | g | 0.1 | g | 0.1 | g | Betaine |
| Ad 100 | g | Ad 100 | g | Ad 100 | g | Water |

### Example 22

### Pump Fixing Foam

| | | |
|---|---|---|
| A | B | C |

| 2 | g | 4 | g | — | | Block polymer no. 65 |
|---|---|---|---|---|---|---|
| 2 | g | — | | 4.0 | g | Block polymer no. 9 |
| 0.3 | g | 0.3 | g | 0.3 | g | Celquat ® L200 |
| 8.9 | g | 8.9 | g | 8.9 | g | Ethanol |
| 0.2 | g | 0.2 | g | 0.2 | g | Cocamidopropyl |
| | | | | | | hydroxysultaine |
| 0.2 | g | 0.2 | g | 0.2 | g | Cetyl trimethyl ammonium |
| | | | | | | chloride |
| 0.15 | g | 0.15 | g | 0.15 | g | Perfume |
| 0.1 | g | 0.1 | g | 0.1 | g | Citric acid |
| 0.1 | g | 0.1 | g | 0.1 | g | Betaine |
| Ad 100 | g | Ad 100 | g | Ad 100 | g | Water |

### Example 23

### Pump Fixing Foam

| A | | B | | C | | |
|---|---|---|---|---|---|---|
| 2.2 | g | 4.4 | g | — | | Block polymer no. 64 |
| 2.2 | g | — | | 4.4 | g | Block polymer no. 8 |
| 2.4 | g | 2.4 | g | 2.4 | g | Advantage ® LC-E¹⁾ |
| 8.9 | g | 8.9 | g | 8.9 | g | Ethanol |
| 0.4 | g | 0.4 | g | 0.4 | g | Cocamidopropyl |
| | | | | | | hydroxysultaine |
| 0.15 | g | 0.15 | g | 0.15 | g | Perfume |
| 0.1 | g | 0.1 | g | 0.1 | g | Citric acid |
| Ad 100 | g | Ad 100 | g | Ad 100 | g | Water |

¹⁾Vinyl caprolactam/VP/dimethylamino ethyl methacrylate copolymer

### Example 24

### Pump Fixing Foam

| A | | B | | C | | |
|---|---|---|---|---|---|---|
| 2.6 | g | 5.1 | g | — | | Block polymer no. 70 |
| 2.6 | g | — | | 5.1 | g | Block polymer no. 14 |
| 0.35 | g | 0.35 | g | 0.35 | g | Luvimer ® 30 E¹⁾ |
| 0.05 | g | 0.05 | g | 0.05 | g | Chitosan (C XII) |
| 0.89 | g | 0.89 | g | 0.89 | g | Aquaflex ® SF 40²⁾ |
| 0.06 | g | 0.06 | g | 0.06 | g | Aminomethyl propanol 95% |
| 8.9 | g | 8.9 | g | 8.9 | g | Ethanol |
| 0.4 | g | 0.4 | g | 0.4 | g | Cocamidopropyl |
| | | | | | | hydroxysultaine |
| 0.15 | g | 0.15 | g | 0.15 | g | Perfume |
| 0.1 | g | 0.1 | g | 0.1 | g | Citric acid |
| Ad 100 | g | Ad 100 | g | Ad 100 | g | Water |

¹⁾t-butyl acrylate/ethyl acrylate/methacrylic acid copolymer
²⁾PVP/vinyl caprolactam/DMAPA acrylates copolymer

In each of the previously mentioned cosmetic hair compositions, another of the block polymers no. 1 to 200 can be used alternatively or in addition.

The abbreviations used in the examples have the following meanings:

PEG-4-k, PEG-6k, PEG-8k: Polyethylene glycol with molecular weight of 4,000, 6,000 or 8,000, respectively.

PDL: Pentadecalactone; lactone of 15-hydroxypentadecanoic acid

PPDL: Poly(pentadecalactone), poly(15-hydroxypentadecanoic acid)

P-LL-LA: Poly(L-lactic acid)

P-DL-LA: Poly(DL-lactic acid)

PCL: Poly(ε-caprolactone)

P(CL-co-LA): Poly(e-caprolactone-co-DL-lactic acid)

PCL-b-PPDL: Poly(e-caprolactone)-block-poly(pentadecalactone)

PEG(4k)-DMA, PEG(8k)-DMA, PEG(10k)-DMA:

poly(ethylene glycol)-dimethacrylate

PLGA(7k)-DMA: Poly(L-lactide-co-glycolide)-dimethacrylate

PCl(10k)-DMA: Poly(ε-caprolactone)-dimethacrylate

D-OEt 1000: Dimerdiol-oligoether Mn=1000 (Sovermol 909)

D-OEt 2000: Dimerdiol-oligoether Mn=2000 (Sovermol 910)

D-OEs 1000: Oligoester-diol Mₙ=1000
from glycerol monostearate and azelaic acid

D-OEs 2000: Oligoester-diol Mₙ=2000
from hydroxystearyl alcohol and adipic acid

D-OEs 3000: Oligoester-diol Mₙ=3000
from hydroxystearyl alcohol and azelaic acid

D-OEs 4000: Oligoester-diol Mₙ=4000
from hydroxystearyl alcohol and azelaic acid

D-OEs 5000: Oligoester-diol Mₙ=5000
from hydroxystearyl alcohol and azelaic acid

1. A method for hair treatment, comprising:
applying to the hair a composition that contains at least one shape memory polymer P and/or at least one cross-linkable macromer M that forms a shape memory polymer after cross-linking is applied to the hair;
wherein polymer P and macromer M are each formed from block polymers having at least a first block which is a polyol selected from polyethers, oligoethers, hydrocarbons having a molecular weight of at least 400 g/mol and at least two alcoholic hydroxyl groups, oligoester diols and polyesters of dicarboxylic acids with diols and at least two additional blocks, which are polyesters of hydroxycarboxylic acids or their lactones;
wherein macromer M comprises:
a) cross-linkable regions that are cross-linkable through chemical bonds,
b) thermoplastic regions that are not chemically cross-linkable, and
c) the shape memory polymer formed after cross-linking has at least one transition temperature Tₜᵣₐₙₛ;
and wherein polymer P comprises:
a) at least one hard segment that can be cross-linked by means of physical interaction, said hard segment having a first transition temperature T′ₜᵣₐₙₛ, which is above room temperature, and
b) at least one soft segment with a second transition temperature Tₜᵣₐₙₛ which is lower than T′trans;
arranging the hair into a defined shape before, simultaneously or subsequently to the step of applying to the hair the composition; and
fixing the defined shape by chemically cross-linking macromer M while forming a shape memory polymer and/or physically cross-linking polymer P.

2. The method according to claim 1, wherein macromer M has the formula A(B—X)ₙ and polymer P has the formula A(B)ₙ, wherein
A is derived from an n-valent polyether or oligoether, hydrocarbons having a molecular weight of at least 400 g/mol and n alcoholic hydroxyl groups, oligoester diols or from a polyester of a dicarboxylic acid with one diol,
B represents a poly(hydroxycarboxylic acid) block,
n represents a number greater than or equal to two, and
X represents a reactive, chemically cross-linkable group.

3. The method according to claim 2, wherein
A is selected from polyalkylene glycol ethers of polyvalent alcohols, poly(tetrahydrofurane), dimerdiol, dimerdiol oligoethers, and oligoester diols,
B is selected from poly(ε-caprolactone), poly(pentadecalactone), polylactides, polyglycolides, and poly(lactide-co glycolide),
X is selected from ethylenically unsaturated, radically polymerizable groups, and
n is 2, 3 or 4.

4. The method according to claim 1, wherein macromer M has the general formula:
X1-0-[B1-C(═O)O-]ₙ₁[Y—O]ₙ₂[C(═O)—B2-O-]ₙ₃X2,
or shape memory polymer P has the general formula:
HO—[B1-C(═O)O-]ₙ₁[Y—O]ₙ₂[C(═O)—B2-O-]ₙ₃H,
wherein
X1 and X2 are the same or different and represent reactive, chemically cross-linkable groups,
B1 and B2 are the same or different and represent branched, cyclic or linear alkylene groups with 1 to 40 C atoms,
Y represents a branched, cyclic or linear alkylene group with 2 to 30 C atoms or for a polyester block of dicarboxylic acid and diol, and
n1, n2 and n3 are the same or different numbers greater than zero.

5. The method according to claim 4, wherein
X1 and X2 are ethylenically unsaturated, radically polymerizable groups,
B1 and B2 represent branched, cyclic or linear alkylene groups with 2 to 20 C atoms, and
Y represents ethylene groups and/or propylene groups.

6. The method according to claim 5, wherein
X1 and X2 are acrylate or methacrylate,
B1 and B2 represent branched or linear alkylene groups with 2 to 20 C atoms,
Y represents an ethylene group, and
n1, n2 and n3 are selected such that the molecular weight of macromer M or polymer P is greater than or equal to 2,000.

7. The method according to claim 1, wherein
the defined shape is heated to a temperature above Tₜᵣₐₙₛ,
the hair is brought into a second shape, and
the second shape is fixed by cooling to a temperature below Tₜᵣₐₙₛ.

8. The method according to claim 7, wherein the fixed second shape is heated to a temperature above Tₜᵣₐₙₛ.

9. The method according to claim 1, wherein the composition additionally contains a macromer with only one chemically reactive group located at a terminal position or a side position.

10. The method according to claim 9, wherein the additional macromer has the general formula:
R—(X′)ₙA3
wherein R represents a monovalent organic residue, A3 represents reactive, chemically cross-linkable group, and —(X′)ₙ— represents a divalent, thermoplastic polymer or oligomer segment.

11. The method according to claim 10, wherein the additional macromer is selected from polyalkylene glycols substituted with an acrylate group or methacrylate group or from its monoalkyl ethers and block copolymers.

12. The method according to claim 1, wherein at least one shape memory polymer P is used and that the shaping of the hair takes place under heating to a temperature of at least T′ₜᵣₐₙₛ and that the subsequent fixing of the hair shape takes place by cooling to a temperature below T′ₜᵣₐₙₛ.

13. The method according to claim 12, wherein shaping of the hair takes place under heating
to a temperature between T′trans and Ttrans,
the hair is brought into a second shape, and
the second shape is fixed by means of cooling to a temperature below T′ₜᵣₐₙₛ.

14. The method according to claim 12, wherein shaping of the hair takes place under heating
to a temperature above T′ₜᵣₐₙₛ,
the hair is brought into a new shape, and
the new shape is subsequently fixed by means of cooling to a temperature below T′ₜᵣₐₙₛ.

15. The method according to claim 1, wherein shape memory polymer P has a degree of crystallinity of from 3 to 80%, and the ratio of the moduli of elasticity below and above Tₜᵣₐₙₛ is at least 20.

16. A cosmetic composition comprising, in a suitable cosmetic foundation, at least one active ingredient selected from macromers M, wherein macromer M comprises:
a) cross-linkable regions that are cross-linkable through chemical bonds,
b) thermoplastic regions that are not chemically cross-linkable, and
c) the shape memory polymer formed after cross-linking has at least one transition temperature Tₜᵣₐₙₛ.

17. The composition according to claim 16, wherein the active ingredient is present in an amount of from 0.01 to 25 percent by weight.

18. The composition according to claim 16, further comprising from 0.01 to 25 percent by weight of at least one additional active ingredient, said active ingredient being selected from macromers with only one chemically reactive group located at a terminal position or a side position, polymers P, hair-care substances, hair-fixing substances and hair-colouring substances, wherein polymer P comprises:
a) at least one hard segment that can be cross-linked by means of physical interaction, said hard segment having a first transition temperature T′ₜᵣₐₙₛ, which is above room temperature, and
b) at least one soft segment with a second transition temperature Tₜᵣₐₙₛ which is lower than T′trans.

19. A cosmetic substance comprising composition according to claim 16, in the form of a lotion, a spray lotion, a cream, a gel, a foam-gel, an aerosol spray, a non-aerosol spray, an aerosol foam, a non-aerosol foam, an o/w or a w/o emulsion, a micro-emulsion or a hair wax.

20. (canceled)
